(19) United States
(12) Patent Application Publication
(10) Pub. No.: US 2022/0152165 A1
(43) Pub. Date: May 19, 2022

(54) COMBINATION OF NASAL GENE DELIVERY AND ORAL CINNAMIC ACID, OLEAMIDE OR GEMFIBROZIL FOR LYSOSOMAL STOARGE DISORDERS

(71) Applicant: Rush University Medical Center, Chicago, IL (US)

(72) Inventor: Kalipada Pahan, Skokie, IL (US)

(21) Appl. No.: 17/441,029

(22) PCT Filed: Mar. 20, 2020
(86) PCT No.: PCT/US2020/023768

§ 371 Date: Sep. 20, 2021

Related U.S. Application Data
(60) Provisional application No. 62/822,310, filed on Mar. 22, 2019.

(51) Int. Cl. A61K 38/48; A61K 48/00; A61K 38/46; A61K 38/47; A61K 38/17; A61K 31/216; A61K 31/192; A61K 31/16; A61K 31/203; A61P 25/28; A61P 3/00

(52) U.S. Cl. CPC A61K 38/4813; A61K 48/00; A61K 38/465; A61K 38/47; A61K 38/1709; A61P 3/00; A61K 31/192; A61K 31/16; A61K 31/203; A61P 25/28; A61K 31/216

(57) ABSTRACT

Provided herein are methods for the treatment of lysosomal storage disease comprising administration of genes encoding for a lysosomal enzyme and a pharmaceutical agent. Combining gene therapy with pharmaceutical compositions by co-administration not only further enhances the effects of each individual therapy, but also provides a multi-faceted approach to treatment because of the varying mechanism of action of each individual composition.

### CROSS-REFERENCE FOR RELATED APPLICATIONS

This application claims priority to U.S. Provisional Patent Application No. 62/822,310 filed Mar. 22, 2019, which is incorporated by reference herein in its entirety.

### TECHNICAL FIELD

The present invention relates to methods of administering genes encoding lysosomal enzymes in combination with pharmaceutical agents for the treatment of lysosomal storage disorders, such as late infantile Batten disease and Krabbe disease.

### BACKGROUND

Lysosomes are membrane bound organelles containing several enzymes that are responsible for the degradation of lipid, protein, carbohydrates, and nucleic acids (De Duve and Wattiaux, 1966). Defects and deficiencies in almost any of these components results in accumulation of undigested and/or partially digested material in the lysosomes, thus forming the basis for numerous lysosomal storage disorders (LSDs) (De Duve and Wattiaux, 1966, Perez-Sala et al., 2009), including Batten disease (infantile, late-infantile and juvenile neuronal ceroid lipofuscinosis), Krabbe disease and Tay-Sachs disease.

Neuronal ceroid lipofuscinosis (NCL) is a group of neurodegenerative diseases primarily composed of typical autosomal recessive lysosomal storage disorders. The NCLs can be characterized by clinical manifestations like progressive mental deterioration, cognitive impairment, visual failures, seizures and deteriorating motor function accompanied by histological findings such as the accumulation of autofluorescent storage material in neurons or other cell types (Hachiya et al., 2006). The NCLs have been subdivided into several groups (Type1-10) based on the age of onset, ultrastructural variations in accumulated storage materials, and genetic alterations unique to each specific disease type (Lane et al., 1996 and Mole et al., 2005).

Infantile neuronal ceroid lipofuscinosis (INCL) presents itself in children at about age 18 months with symptoms including blindness, cognitive defects, seizures and early death (Hawkins-Salsbury et al., 2013). Defects in the cln1 gene encoding for the lysosomal enzyme palmitoyl protein thioesterase-1 (PPT1) causes the accumulation of various autofluorescent material substrates, such as liposfuscin, in both the central nervous system and tissues (Id.). The ensuing neuronal degeneration, cortical thinning and brain atrophy results in an approximately 50% reduction of brain mass as compared to the unaffected child (Id.). Currently no treatments are available.

Late infantile neuronal ceroid lipofuscinosis (Jansky-Bielschowsky disease, LINCL, Type 2) typically produces symptoms at the age of 2-4 years, progresses rapidly and ends in death between ages 8 to 15 as a result of a dramatic decrease in the number of neurons and other cells (Lane et al., 1996 and Sleat et al., 1997). LINCL is associated with mutations in the cln2 gene, a 13 exon and 12 intron gene of total length of 6.65 kb mapped to chromosome 11p15.5. The cln2 gene encodes lysosomal tripeptidyl tripeptidase I (TPP-I or pepstin insensitive protease), a 46 KD protein that functions in the acidic environment of the lysosomal compartment to remove tripeptides from the amino terminus of proteins (Goebel 1995 and Vines et al., 1999). This mutation in the cln2 gene results in a deficiency and/or loss of function of the TPP1 protein that leads to intra-lysosomal accumulation of autofluoroscent lipopigments known as ceroid-lipofuscin (Goebel, 1995). Currently there is no established treatment or drugs available for this disease and all approaches are merely supportive or symptomatic, indicating a need for novel therapeutic approaches (Chang et al., 2008). However, there are different variants of cln2 mutations and there have been reports that residual TPP-I activity can be found in patients with LINCL, indicating that there must be a few copies of normal cln2 gene remaining in patients affected with LINCL (Viglio et al., 2001 and Walus et al., 2010).

Another NCL is juvenile Batten disease (juvenile infantile neuronal ceroid lipofuscinosis (JINCL)). The cln3 gene encodes for a lysosomal transmembrane protein that may be involved in synapse function or degradation (Dolisca et al., 2013). The mutation in cln3 associated with JINCL is characterized by a 1.01 kb deletion. As with the other NCL's, the onset of JINCL occurs in children between the ages of 4 and 7 with symptoms including gradual blindness, motor and cognitive deterioration, seizures and early death.

Krabbe disease is a rare lysosomal storage disease and is the result of sphingolipidoses based deterioration of the myelin sheath. The disease is caused by a mutation in the β-galactocerebrosidase lysosomal storage enzyme, whereby cytotoxic metabolites accumulate and disrupt various metabolic pathways that result in demyelination. Krabbe disease can be infantile, late-infantile, juvenile and even adult form (Pavuluri et al., 2017).

Tay-Sachs disease is the result of mutations in the hexa gene, which encodes for β-hexosaminidase, the enzyme responsible for processing of GM2 ganglioside to GM3 gangliosyde (Dersh et al., 2016). The enzyme is made of two subunits and the mutation results in loss or inactivity of the enzyme resulting in accumulation of GM2. There are over 100 mutations that have been identified in the hexa gene associated with Tay-Sachs disease (Id.).

Because various genetic mutations are associated with multiple enzymes resulting in lysosomal storage disorders, gene therapy is a potential treatment option. However, gene delivery especially for the treatment of neurodegenerative disease presents the problem of delivering therapeutic genes to the brain. Viral based gene delivery mechanisms are well known and gene delivery can be accomplished specifically through the use of Adeno-associated viral vectors because of the poor immunogenicity of the virus (Shaw et al., 2013). Furthermore, nasal administration of these viral vectors comprising therapeutic genes allows for delivery to the brain. Nasal delivery is believed to take advantage of the “nose-to-brain” (N2B) transport systems (Djupesland, 2013) in which several possibilities exist for bypassing the blood-brain-barrier for direct delivery to the brain. These include the draining of drugs absorbed in the nasal mucosa into the sinus and eventually to the carotid artery, where a “counter-current transfer” from venous blood to the brain may occur. Lymphatic drainage into the perivascular space from the olfactory trigeminal nerves between the central nervous system (CNS) have also been postulated as the mechanism of N2B transport.

Furthermore, combining gene therapy with oral pharmaceuticals treatment provides another powerful therapeutic approach that enhances the effects of mono therapies. Two pharmaceutical agent candidates for combination with gene therapy include cinnamic acid and oleamide. Cinnamic acid is a naturally occurring fatty acid found in plants with neuroprotective effects (Prorok et al., 2019). It has been found to be involved in the activation of peroxisome proliferator-activated receptorα (PPARα) for the protection of dopaminergic neurons in Parkinson's Disease (Id.) Various derivatives of cinnamic acid are also known for their antioxidant profile and the ability to cross the blood-brain barrier, which makes these agents ideal for treating neurodegenerative disorders (Roleira et al., 2010). Oleamide is another fatty acid with a wide range of neuropharmacological actions. A known, endogenous fatty acid, oleamide was first found in cerebrospinal fluid (Nam et al., 2017). It is constitutively present in the hippocampus where it acts a PPARα ligand and is involved in inducing sleep (Pahan, 2017). Thus, the potential use of cinnamic acid as a natural pharmaceutical agent and oleamide as an endogenous brain ligand in combination with gene therapy is merited.

In addition, several studies have concluded that neuro-inflammation and induction of apoptotic pathways can be attributed to the neuronal damage in most forms of NCL, including LINCL (Geraets et al. 2016, Dhar et al. 2002, Puranam et al. 1997, Kohan et al. 2011). Although inflammation is not the initiating factor in LINCL, glia-mediated sustained inflammatory response is believed to contribute to disease progression (Cooper et al. 2015, Macauley et al. 2014). Gemfibrozil, an FDA-approved lipid-lowering drug, is known to reduce the level of triglycerides in the blood circulation and decrease the risk of hyperlipidemia (Robins et al. 2001, Rubins & Robins 1992, Rubins et al. 1999). However, a number of recent studies reveal that apart from its lipid-lowering effects, gemfibrozil can also regulate many other signaling pathways responsible for inflammation, switching of T-helper cells, cell-to-cell contact, migration, oxidative stress, and lysosomal biogenesis (Ghosh & Pahan 2012a, Corbett et al. 2012, Ghosh et al. 2012, Jana et al. 2007, Jana & Pahan 2012, Dasgupta et al. 2007, Pahan et al. 2002, Roy & Pahan 2009, Ghosh et al. 2015). Thus, gene therapy in combination with gemfibrozil also has great potential.

### SUMMARY

One embodiment described herein is a method for treatment of a lysosomal storage disease comprising administering to a subject in need thereof a first composition comprising a therapeutically effective amount of a gene encoding for a lysosomal enzyme and a second composition comprising a therapeutically effective amount of a pharmaceutical agent.

In one aspect, the first composition is administered intra-nasally.

In another aspect, the gene is delivered across the blood, brain barrier.

In another aspect, the first composition is administered about once every 7-30 days.

In yet another aspect, the first composition comprises a viral vector comprising the gene encoding for a lysosomal enzyme.

In one aspect, the viral vector is an adenovirus-associated viral vector.

In another aspect, the gene comprises ppt1, cln2, cln3, galc, or hexa.

In another aspect, the lysosomal enzyme comprises palmitoyl-protein thioesterase-1, tripeptidyl peptidase 1, galactosylceramide, battenin or hexosaminidase A.

In a yet another aspect, the method comprises administering the first composition comprising the ppt1 gene for treating the lysosomal storage disease comprising Infantile Neuronal Ceroid Lipofuscinosis.

In one aspect, the method comprises administering the first composition comprising the cln2 gene for treating the lysosomal storage disease comprising Late Infantile Neuronal Ceroid Lipofuscinosis.

In another aspect, the method comprises administering the first composition comprising the cln3 gene for treating the lysosomal storage disease comprising Juvenile Neuronal Ceroid Lipofuscinosis.

In another aspect, the method comprises administering the first composition comprising the galc gene for treating the lysosomal storage disease comprising Krabbe disease.

In yet another aspect, the method comprises administering the first composition comprising the hexa gene for treating the lysosomal storage disease comprising Tay-Sachs disease.

In one aspect, the pharmaceutical agent comprises cinnamic acid, oleamide or fibrate.

In another aspect, the fibrate is gemfibrozil or fenofibrate.

In another aspect, the second composition further comprises a therapeutically effective amount of all-trans retinoic acid.

In yet another aspect, the therapeutically effective amount of the pharmaceutical agent is lower when the pharmaceutical agent is administered in combination with all-trans retinoic acid than when the pharmaceutical agent is delivered without all-trans retinoic acid.

In one aspect, the second composition is administered orally.

In another aspect, the second composition is administered once daily.

In another aspect, administering the first composition and the second composition provides a greater therapeutic effect in the subject than administration of the first composition or the second composition alone.

In yet another aspect, the lysosomal storage disorder is selected from the group consisting of late-infantile Batten disease, juvenile Batten disease, Krabbe disease, Tay-Sachs disease, Niemann-Pick disease, Fabry disease, Farber disease and Gaucher disease.

In one aspect, the first composition is administered intra-nasally and the second composition is administered orally.

In another aspect, the first composition is administered at least once every 7 days and the second composition is administered once daily.

In another aspect, the viral vector is an adenovirus-associated viral vector.

In another aspect, the gene comprises cln2.

In another aspect, the second composition comprises gemfibrozil.

In another aspect, the administration of the first composition increased lifespan by about 100 days.

### BRIEF DESCRIPTION OF THE DRAWINGS

FIG. 1: Intranasal delivery of adenoviral human Cln2 gene (Ad-Cln2) prolongs the life span of Cln2^{(-/-)} mice, an animal model of late infantile Batten disease. For intranasal gene delivery, Cln2^{(-/-)} mice received 5×10⁶ genome copies of Ad-Cln2 in a volume of 5 l twice a week intranasally (2.5 μl/nostril) starting from two weeks of age for four weeks. For gemfibrozil (gem) treatment, mice received gem (dissolved in 0.1% MeC) orally at a dose of 7.5 mg/kg body weight/day starting from six weeks of age. FIG. 1 describes the percentage of survival is shown by Kaplan-Meier plot.

FIG. 2: Intranasal delivery of adenoviral human Cln2 gene (Ad-Cln2) prolongs the life span of Cln2^{(-/-)} mice, an animal model of late infantile Batten disease. For intranasal gene delivery, Cln2^{(-/-)} mice received 5×10⁶ genome copies of Ad-Cln2 in a volume of 5 l twice a week intranasally (2.5 μl/nostril) starting from two weeks of age for four weeks. For gemfibrozil (gem) treatment, mice received gem (dissolved in 0.1% MeC) orally at a dose of 7.5 mg/kg body weight/day starting from six weeks of age. FIG. 2 describes mean survival days. Six mice (n=6) containing 3 males and 3 females were used in each group. ***p<0.001; NS, not significant.

### DETAILED DESCRIPTION

The embodiments disclosed herein are not intended to be exhaustive or to limit the scope of the disclosure to the precise form in the following description. Rather, the embodiments are chosen and described as examples herein so that others skilled in the art may utilize their teachings.

The present disclosure relates to methods of co-administering genes encoding lysosomal enzymes in combination with pharmaceutical agents for the treatment of lysosomal storage disorders, such as late infantile Batten disease and Krabbe disease.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The term “intra-nasal”, as used herein, refers to modes of administration which include contact with the nasal mucosal surfaces or inhalation for absorption in the bronchial passages of the lungs.

The term “oral”, as used herein, refers to modes of administration which include oral, enteral, buccal, sublabial, and sublingual gastric administration.

“Treating”, “treat”, or “treatment” as used herein, means an alleviation of symptoms associated with a disorder or disease, or halt of further progression or worsening of those symptoms, or prevention or prophylaxis of the disease or disorder. For example, within the context of this disclosure, successful treatment may include prevention of a neurodegenerative disease, an alleviation of symptoms related to neurodegenerative disease or a halting in the progression of a disease such as a neurodegenerative disease. As used herein, a control for measuring the treatment relative it a control is a subject that has not received the therapeutic agent.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Provided herein are methods of co-administration of a gene encoding for a lysosomal enzyme and a pharmaceutical composition to a subject comprising administering a therapeutically effective amount of the gene and pharmaceutical agent for the treatment of lysosomal storage disorders.

Gene Compositions

In one embodiment described herein are gene compositions which may include a “therapeutically effective amount” of the therapeutic gene of interest. A “therapeutically effective amount” refers to an amount effective, at dosages and for periods of time necessary, to achieve the desired therapeutic result. A therapeutically effective amount of the therapeutic gene may be determined by a person skilled in the art and may vary according to factors such as the disease state, age, sex, and weight of the individual, and the ability of the composition to elicit a desired response in the individual. A therapeutically effective amount is also one in which any toxic or detrimental effects of the gene are outweighed by the therapeutically beneficial effects.

In one aspect described herein, the method for delivering a composition comprising a therapeutic gene is via intra-nasal administration. Methods of delivering compositions comprising therapeutic genes include any number of modes of administration to the nose including delivery of liquid or powder formulations of compositions for nasal administration via either passive of active delivery mechanisms. In one embodiment, liquid formulations, may be delivered through a variety of mechanisms including vaporization through nasal inhalation, hand actuated nasal devices and mechanical spray pumps. In another embodiment, formulations for such delivery mechanisms may be in the form of propellant containing aerosols or propellant-free inhalable solutions. In another embodiment, mechanical spray pumps may be hand actuated, gas driven or electrical, as in the case of electrically powered nebulizers and atomizers. In a further embodiment, powder formulations may be delivered though mechanical power sprayers, nasal inhalers and nebulizers/atomizers.

The goal of intra-nasal administration is for ultimate delivery of the therapeutic gene across the blood-brain-barrier to the brain. Without being bound by any theory, nasal administration of gene therapies can take advantage of “nose-to-brain” (N2B) transport systems (Djupesland, 2013) in which several possibilities exist for bypassing the blood-brain-barrier for direct delivery to the brain. These include the draining of drugs absorbed in the nasal mucosa into the sinus and eventually to the carotid artery, where a “counter-current transfer” from venous blood to the brain may occur. Thus, in one aspect described herein, the gene is delivered across the blood, brain barrier.

In one embodiment described herein, a gene composition comprising a therapeutically effective amount of a gene is administered once about every 1 to about every 100 days, once about every 2 to about every 90 days, once about every 3 to about every 80 days, once about every 4 to about every 70 days, once about every 5 to about 60 days, once about every 6 to about 50 days, once every 7 to about 40 days, once about every 8 to about every 30 days, or once about every 9 to about 20 days. In one aspect, the gene composition is administered once about every 7 to about every 30 days. In another aspect, the gene composition is administered once about every 7 days.

In one embodiment described herein, the therapeutic gene is delivered through the use of a viral vector. Ideal viral vectors for gene therapy can successfully infect the target cell, transfer to the nucleus and maintain expression levels without inducing toxicity. Viral vectors may be comprised of any virus suitable for gene therapy including retroviruses or adenoviruses. Other viruses suitable for viral vectors include adeno-associated viruses, lentiviruses, pox viruses, alphaviruses and herpes viruses. Adeno-associated viral vectors are ideal vectors because of their relatively low pathogenicity and sustained expression. Thus, in one aspect described herein, the viral vector comprises an adeno-associated viral vector.

In another embodiment described herein, the viral vector comprises a therapeutic gene encoding for a lysosomal enzyme. Genes encoding for a lysosomal enzyme and associated proteins include aspartylglucosaminidase (aga), arylsulfatase A (ansa), arylsulfatase B (arsb), acid ceramidase (asah1), autophagy protein 5 (atg5), autophagy protein 7 (atg7), palmitoyl protein thioesterase 1 or PPT1 (c1n1), tripeptidyl peptidase 1 (c1n2), battenin (c1n3), transmembrane endoplasmic reticulum protein (c1n6), endoplasmic reticulum cargo receptor (c1n8), cystinosin (ctns), cathepsin A (ctsa), cathepsin K (ctsk), phosphoinositide phosphatase (fig4), alpha-L-fucosidase 1 (fuca1), acid alpha-glucosidase (gaa), galactosylceramidase (galc), galactosamine (N-acetyl)-6-sulfatase (galns), beta-glucocerebrosidase (gba), alpha-galactosidase A (gla), beta-galactosidase 1 (glb1), GM2 ganglioside activator (gm2a), glcNAc-1-phosphotransferase (gnptab), N-acetylglucosamine-1-phosphotransferase (gnptg), N-acetylglucosamine-6-sulfatase (gns), beta-glucuronidase (gusb), beta-hexosaminidase A (hexa), beta-hexosaminidase B (hexb), heparan-alpha-glucosaminide N-acetyltransferase (hgsnat), hyaluronidase-1 (hyal1), iduronate 2-sulfatase (ids), alpha-L- iduronidase (idua), lysosomal associated membrane protein 2 (lamp2), lysosomal acid lipase (lipa), alpha-mannosidase (man2b 1), beta-mannosidase (manba), mucolipin-1 (mcoln1), mammalian target of rapamycin complex 1 or mechanistic target of rapamycin complex 1 (mtorc1), alpha-N-acetylgalactosaminidase (naga), alpha-N-acetylglucosaminidase (naglu), neuraminidase 1 (neu1), Niemann-Pick C1 (npc1), Niemann-Pick C1 (npc2), patatin-like phospholipase domain-containing protein 1 (pnpla2), palmitoyl-protein thioesterase 1 (ppt1), prosaposin (psap), N-sulfoglucosamine sulfohydrolase (sgsh), sialin protein (slc17a5), TOR regulating protein (slc389), sodium/hydrogen exchanger 6 (slc9A6), acid sphingomyelinase (smpd1), formylglycine-generating enzyme (sumf1), or tripeptidyl peptidase 1 (tpp1). In one aspect described herein, the therapeutic gene comprises ppt1, cln2, cln3, galc or hexa.

The lysosomal enzymes responsible for lysosomal storage diseases are vast. Examples of lysosomal enzymes implicated in lysosomal storage disease include α-N-acetylgalactosaminidase, acid ceramidase, acid maltase, acid sphingomyelinase, acid sphingomyelinase, acid β-glucosidase, adipose triglyceride lipase, arylsulfatase A, arylsulfatase B, ATG5, ATG7, battenin, cathepsin K, cystinosin, epididymal secretory protein HE1, galactosamine-6-sulfate sulfatase, galactosylceramide, gamma subunit of N-acetylglucosamine-1-phosphotransferase, glycosylasparaginase, GM2-activator protein, heparan N-sulfatase, hexosaminidase A and B, hyaluronidase, iduronate 2-sulfatase, lysosomal acid lipase, lysosomal β-mannosidase, lysosome-associated-membrane protein-2, monovalent sodium-selective sodium/hydrogen exchanger (NHE), mTORC1, mucolipin-1, N-α-acetylglucosaminidase, neuraminidase, palmitoyl-protein thioesterase-1, PIP(2) 5-phosphatase, protective protein/cathespin A. saposin B, saposin C, sialin, SLC38A9, sulfatase-modifying factor-1, tripeptidyl peptidase 1, α-galactosidase, α-L-fucosidase, α-L-iduronidase, α-mannosidase, or β-glucosidase. In one aspect described herein, the lysosomal enzyme comprises palmitoyl-protein thioesterase-1, tripeptidyl peptidase 1, galactosylceramide, battenin, or hexosaminidase A.

Intra-nasal delivery of therapeutic genes for targeting the brain is ideal for the treatment of neurodegenerative and lysosomal storage disorders. Neurodegenerative disorder may include Alzheimer's disease (AD), Huntington's disease, Amyotrophic lateral sclerosis (ALS), Parkinson's disease, including Parkinson's plus diseases such as multiple system atrophy (MSA), multiple sclerosis (MS), progressive supranuclear palsy (PSP), corticobasal degeneration (CBD) or dementia with Lewy bodies (DLB). The neurodegenerative disease may be caused by a lysosomal storage disorder. Batten disease is the most common form of a group of disorders called the neuronal ceroid lipofuscinosis (NCL), including Infantile Neuronal Ceroid Lipofuscinosis (INCL), Late Infantile Neuronal Ceroid Lipofuscinosis (LINCL), and Juvenile Neuronal Ceroid Lipofuscinosis (JINCL). The lysosomal storage disorder may also be, for example, Tay-Sach's disease, Fabry disease, Niemann-Pick disease, Krabbe disease, Gaucher disease, Hunter Syndrome, Alpha-mannosidosis, Aspartylglucosaminuria, Cholesteryl ester storage disease, Chronic Hexosaminidase A Deficiency, Cystinosis, Danon disease, Farber disease, Fucosidosis, or Galactosialidosis. In one aspect, the lysosomal storage disorder comprises Infantile Neuronal Ceroid Lipofuscinosis (INCL), Late Infantile Neuronal Ceroid Lipofuscinosis (LINCL), and Juvenile Neuronal Ceroid Lipofuscinosis (JINCL) or Krabbe disease. In one aspect described herein, the lysosomal storage disorder comprises late-infantile Batten disease, juvenile Batten disease, Krabbe disease, Tay-Sachs disease, Niemann-Pick disease, Fabry disease, Farber disease and Gaucher disease.

In another aspect described herein, the ppt1 gene encodes an enzyme involved in Infantile Neuronal Ceroid Lipofuscinosis. In another aspect, the cln2 gene encodes an enzyme involved in Late Infantile Neuronal Ceroid Lipofuscinosis. In another aspect, the cln3 gene encodes an enzyme involved in Juvenile Neuronal Ceroid Lipofuscinosis. In yet another aspect, the galc gene encodes an enzyme involved in Krabbe disease. In another aspect, the hexa gene encodes an enzyme involved in Tay-Sachs disease.

Pharmaceutical Compositions

The pharmaceutical compositions may include a “therapeutically effective amount” or a “prophylactically effective amount” of a pharmaceutical agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of the agent are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical agent may be any active ingredient that induces a therapeutic effect for the treatment of lysosomal storage disorders. Agents may be naturally occurring or synthetic. Examples of naturally occurring agents include natural saturated fatty acids and their derivatives, for example, stearic acid, palmitic acid, cinnamic acid, lauric acid, capric acid, and the like. Examples of naturally occurring unsaturated fatty acids and their derivatives include oleic acid, oleamide, linoleic acid, linolenic acid, and ricinoleic acid. In one aspect described herein the pharmaceutical agent is cinnamic acid or oleadmide.

Examples of synthetic agents as the pharmaceutical agent include, for example, lipid-lowering drug such as a fibrate. Non-limiting examples of fibrates include gemfibrozil, fenofibrate, clofibrate, bezafibrate, ciprofibrate and clinofibrate. Gemfibrozil (5-(2,5-dimethylphenoxy)-2,2-dimethylpentanoic acid) is commercially available under the trademark Lopid® by Pfizer. Fenofibrate (2-(4-(4-chlorobenzoyl)phenoxy)-2-methyl-propanoic acid 1-methyl ethyl ester) is available commercially as Tricor® by Abbvie. Additional fibrates include Clofibrate (2-(4-chlorophenoxy)-2-methyl-propanoic ethyl ester), Bezafibrate (2-(4-(2-(4-chloro-benzoylamino)-ethyl)phenoxy)-2-methyl-propanoic acid), Ciprofibrate (2-(4-(2,2-dichlorocyclopropyl)phenoxy)-2-methyl propanoic acid) and Clinobibrate (2-[4-[1-[4-(2-carboxybutan-2-yloxy)phenyl]cyclohexyl]phenoxy]-2-methylbutanoic acid). In one aspect described herein, the pharmaceutical agent is a fibrate. In another aspect described herein, the pharmaceutical agent is gemfibrozil or fenofibrate.

The agent may be incorporated into pharmaceutical compositions suitable for administration to a subject (such as a patient, which may be a human or non-human).

The pharmaceutical composition may further comprise other therapeutically effective agents. In one aspect described herein, the pharmaceutical composition further comprises a therapeutically effective amount of all-trans retinoic acid. All-trans retinoic acid has been implicated in cognitive activities, and has been suggested to reduce oxidative stress associated with Alzheimer's disease (Lee et al., 2009). Thus, administering all-trans retinoic acid with the pharmaceutical agent and the therapeutic gene may provide a further enhanced therapeutic effect in the subject than administration of all-trans retinoic acid, the pharmaceutical agent, or the therapeutic gene alone. In another aspect described herein, the therapeutically effective amount of the pharmaceutical agent is lower when the pharmaceutical agent is administered in combination with all-trans retinoic acid than when the pharmaceutical agent is delivered without all-trans retinoic acid.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Methods of treating neurological diseases such as late infantile neuronal ceroid lipofuscinosis may include any number of modes of administering the pharmaceutical agent or pharmaceutical compositions of the agent. In one aspect described herein, the pharmaceutical composition is administered with the gene composition.

In another aspect described herein, the pharmaceutical composition is administered orally. Oral administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire™). In the pharmaceutical composition, the agent may also be dispersed in a microparticle, e.g. a nanoparticulate, composition.

In one embodiment, a therapeutically effective amount of a pharmaceutical composition is administered once daily about every 1 to about 100 days, once daily about every 2 to about every 90 days, once daily about every 3 to about every 80 days, once daily about every 4 to about every 70 days, once daily about every 5 to about 60 days, once about every 6 to about 50 days, once every 7 to about 40 days, once about every 8 to about every 30 days, or once about every 9 to about 20 days. In another embodiment described herein, the pharmaceutical composition is administered twice about every 1 to about 100 days, twice about every 2 to about every 90 days, once about every 3 to about every 80 days, once about every 4 to about every 70 days, once about every 5 to about 60 days, once about every 6 to about 50 days, once every 7 to about 40 days, once about every 8 to about every 30 days, or once about every 9 to about 20 days. In one aspect described herein the pharmaceutical composition is administered daily.

Combination Therapy

Combining gene therapy with pharmaceutical compositions by co-administration not only further enhances the effects of each individual therapy, but also provides a multi-faceted approach to treatment because of the varying mechanism of action of each individual composition. In this way, not only is enzyme function restored, but the population of functional enzymes is enhanced. Thus, in one aspect described herein, gene delivery restores lysosomal function, and the pharmaceutical agent increases an amount of the lysosomes. In another aspect described herein, co-administration of the first composition and the second composition provides a greater therapeutic effect in the subject than administration of the first composition or the second composition alone. In some aspects, the gene composition may be delivered at one interval and the pharmaceutical composition may be delivered at a second, different interval. In some aspects, the gene composition may be delivered less frequently than the pharmaceutical composition. By way of non-limiting example, the gene composition may be delivered weekly and the pharmaceutical composition may be delivered daily. Other combination dosing regimens may also be used to deliver a combination therapy.

The present disclosure has multiple aspects, illustrated by the following non-limiting examples.

### Examples

Studies will be performed to evaluate the effects intra-nasal gene therapy in combination with pharmaceutical compositions in animal models of neurodegenerative disorders.

Gene and pharmaceutical compositions. Gene compositions will be prepared using adeno-associated viral vectors comprising the ppt1, cln2, cln3, galc or hexa gene. Oral gemfibrozil, cinnamic acid or oleamide compositions will be used.

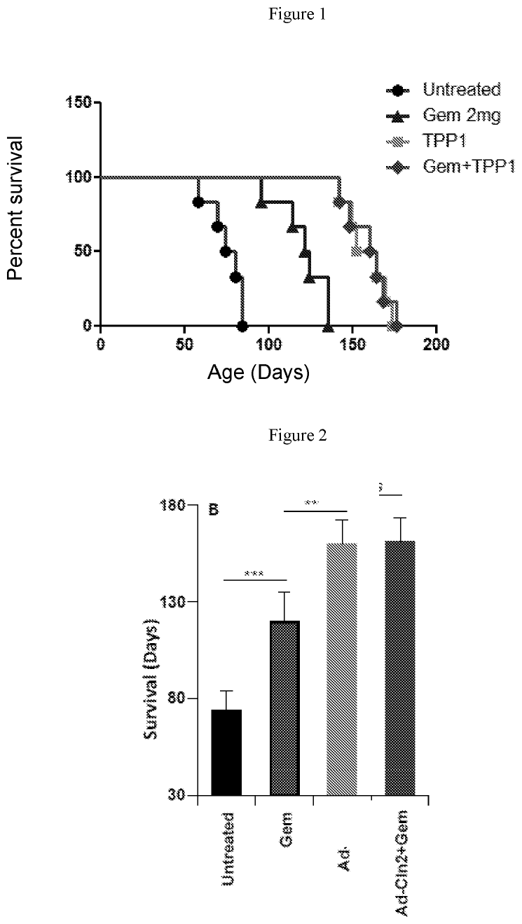

Nebulization: In some aspects, nebulization will be used for intra-nasal delivery, although other intra-nasal methods may also be used, such as, but not limited to nose drops, ointments, atomization pump, and pressurized aerosol. A Buxco Inhalation Tower All-In-One Controller by DSI™ will be used for air supply for nebulization (FIG. 1A). A whole body chamber will be fitted with an Aeroneb® Ultrasonic Nebulizer (FIG. 1B) supplied with air from a Buxco bias flow pump. Mice will nebulize the gene composition at appropriate doses (solubilized in a volume of 100 μl double-distilled water/mouse) for 3 min. The control group of mice will also receive 100 μl water by nebulization.

### Example 1: Infantile Neuronal Ceroid Lipofuscinosis

Treatment of ppt1^{(-/-)} mice with intra-nasal gene therapy and oral gemfibrozil, cinnamic acid or oleamide in mice with INCL: Age- and sex-matched ppt1^{(+/+)} mice from the same background will be used as wild type (WT) controls and ppt1^{(-/-)} animals will be used in different treatment groups. Mice will be treated with the gene therapy composition and the pharmaceutical composition selected from the group consisting of gemfibrozil, cinnamic acid and oleamide and the control group will be treated with carrier only.

ppt1^{(-/-)} mice and controls will be treated with nasal AAV1-PPT1 (2 μl containing 2×10⁶ genome copies per mouse) weekly+oral cinnamic acid (25 mg/kg body wt/d), oleamide (5 mg/kg body wt/d) or gemfibrozil (8 mg/kg body wt/d) daily followed by recording longevity and monitoring storage materials in the brain.

### Example 2: Juvenile Batten Disease

Cln3^{(-/-)} mice will be treated with nasal AAV1-CLN3 (2 μl containing 2×10⁶ genome copies per mouse) weekly+oral cinnamic acid (25 mg/kg body wt/d), oleamide (5 mg/kg body wt/d) or gemfibrozil (8 mg/kg body wt/d) daily followed by recording longevity and monitoring storage materials in the brain.

### Example 3: Krabbe Disease

Galc^{(-/-)} mice will be treated with nasal AAV1-GALC (2 μl containing 2×10⁶ genome copies per mouse) weekly+oral cinnamic acid (25 mg/kg body wt/d), oleamide (5 mg/kg body wt/d) or gemfibrozil (8 mg/kg body wt/d) daily followed by recording longevity and monitoring storage materials in the brain.

### Example 4: Tay-Sachs Disease

Hexa^{(-/-)} mice will be treated with nasal AAV1-HEXA (2 μl containing 2×10⁶ genome copies per mouse) weekly+oral cinnamic acid (25 mg/kg body wt/d), oleamide (5 mg/kg body wt/d) or gemfibrozil (8 mg/kg body wt/d) daily followed by recording longevity and monitoring storage materials in the brain.

### Example 5: Late Infantile Neuronal Ceroid Lipofuscinosis (LINCL)

Intranasal gene delivery was examined as a valid option for fatal lysosomal storage disorders. A mouse model of Late Infantile Neuronal Ceroid Lipofuscinosis (LINCL) was used, a rare neurodegenerative disease caused by mutations in the Cln2 gene that leads to deficiency or loss of function of the tripeptidyl peptidase 1 (TPP1) enzyme.

An adenoviral vector was delivered of human Cln2 gene (Ad-Cln2) to two weeks old Cln2^{-/-} mice via intranasal route (5×10⁶ genome copies of Ad-Cln2 in a volume of 5 μl twice a week; 2.5 μl/nostril). After 4 weeks of intranasal gene therapy, one group of mice (n=6) left untreated and the other group of mice (n=6) were treated with gemfibrozil orally at a dose of 7.5 mg/kg body weight/day. Therefore, one group of Cln2^{-/-} mice not receiving Ad-Cln2 were also treated with gemfibrozil orally.

It was found that gemfibrozil treatment significantly increased the lifespan of Cln2^{-/-} mice (FIGS. 1-2). However, four weeks of biweekly intranasal gene delivery alone was significantly more effective than gemfibrozil in increasing the life span of Cln2^{-/-} mice (FIGS. 1-2). However, four weeks of biweekly intranasal gene delivery alone was significantly more effective than gemfibrozil in increasing the life span of Cln2^{-/-} mice (FIGS. 1-2). In contrast, oral gemfibrozil treatment did not further increase the lifespan of Cln2^{-/-} mice that received intranasal Ad-Cln2 (FIGS. 1-2).

All publications, patents and patent applications cited in this specification are incorporated herein by reference for the teaching to which such citation is used.

The specific responses observed may vary according to and depending on the particular type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

### REFERENCES

Chang, M., Cooper, J. D., Sleat, D. E., Cheng, S. H., Dodge, J. C., Passini, M. A., Lobel, P., and Davidson, B. L. (2008) Mol Ther 16, 649-656.
Cooper, J. D., Tarczyluk, M. A. and Nelvagal, H. R. (2015) Towards a new understanding of NCL pathogenesis. Biochim Biophys Acta, 1852, 2256-2261.
Corbett, G. T., Gonzalez, F. J. and Pahan, K. (2015) Activation of peroxisome proliferator-activated receptor alpha stimulates ADAM10-mediated proteolysis of APP. Proc Natl Acad Sci USA, 112, 8445-8450.
Dasgupta, S., Roy, A., Jana, M., Hartley, D. M. and Pahan, K. (2007) Gemfibrozil ameliorates relapsing-remitting experimental autoimmune encephalomyelitis independent of peroxisome proliferator-activated receptor-alpha. Mol Pharmacol, 72, 934-946.
De Duve, C. and Wattiaux, R. (1966) Functions of lysosomes. Annu Rev Physiol, 28, 435-492.
Dersh, D., Iwamoto, Y., and Argon, Y., Mol Blot Cell (2016) December 1; 27(24): 3813-3827.
Djupesland, P. G. (2013) Nasal drug delivery devices: characteristics and performance in a clinical perspective—a review. Drug Deliv Transl Res. 3(1), 42-62.
Dhar, S., Bitting, R. L., Rylova, S. N., Jansen, P. J., Lockhart, E., Koeberl, D. D., Amalfitano, A. and Boustany, R. M. (2002) Flupirtine blocks apoptosis in batten patient lymphoblasts and in human postmitotic CLN3- and CLN2-deficient neurons. Ann Neurol, 51, 448-466.
Dolisca, S. B., Mehta, M., Pearce, D. A., Mink, J. W., Maria, B. L., J Child Neurol (2013) September; 28(9): 1074-1100.
Food and Drug Administration-approved lipid-lowering drugs, up-regulate tripeptidyl-peptidase 1 in brain cells via peroxisome proliferator-activated receptor alpha: implications for late infantile Batten disease therapy. J Blol Chem, 287, 38922-38935.
Geraets, R. D., Koh, S., Hastings, M. L., Kielian, T., Pearce, D. A. and Weimer, J. M. (2016) Moving towards effective therapeutic strategies for Neuronal Ceroid Lipofuscinosis. Orphanet J Rare Dis, 11, 40.
Goebel, H. H. (1995) J Child Neurol 10, 424-437.
Ghosh, A., Jana, M., Modi, K., Gonzalez, F. J., Sims, K. B., Berry-Kravis, E. and Pahan, K. (2015) Activation of peroxisome proliferator-activated receptor alpha induces lysosomal biogenesis in brain cells: implications for lysosomal storage disorders. J Blol Chem, 290, 10309-10324.
Ghosh, A. and Pahan, K. (2012a) Gemfibrozil, a lipid-lowering drug, induces suppressor of cytokine signaling 3 in glial cells: implications for neurodegenerative disorders. J Blol Chem, 287, 27189-27203.
Hachiya, Y., Hayashi, M., Kumada, S., Uchiyama, A., Tsuchiya, K., and Kurata, K. (2006) Acta Neuropathol 111, 168-177.
Hawkins-Salsbury, J. A., Cooper, J. D., and Sands, M. S., (2013) Biochim Biophys Acta 1832(11): 1906-1909.
Jana, M. and Pahan, K. (2012) Gemfibrozil, a lipid lowering drug, inhibits the activation of primary human microglia via peroxisome proliferator-activated receptor beta. Neurochem Res, 37, 1718-1729.
Kohan, R., Cismondi, I. A., Oller-Ramirez, A. M., Guelbert, N., Anzolini, T. V., Alonso, G., Mole, S. E., de Kremer, D. R. and de Halac, N. I. (2011) Therapeutic approaches to the challenge of neuronal ceroid lipofuscinoses. Curr Pharm Biotechnol, 12, 867-883.
Lane, S. C., Jolly, R. D., Schmechel, D. E., Alroy, J., and Boustany, R. M. (1996) Neurochem 67, 677-683.
Lee, H. P., Casadesus, G., Zhu, X., Lee, H., Perry, G., Smith, M. A., Gustaw-Rothenberg, K. and Lerner, A. (2009) Expert Rev Neurother. 9(11): 1615-1621.
Macauley, S. L., Wong, A. M., Shyng, C. et al. (2014) An anti-neuroinflammatory that targets dysregulated glia enhances the efficacy of CNS-directed gene therapy in murine infantile neuronal ceroid lipofuscinosis. J Neurosci, 34, 13077-13082.
Mole, S. E., Williams, R. E., and Goebel, H. H. (2005) Neurogenetics 6, 107-126.
Nam, H. Y., Na, E. J., Lee, E., Kwon, Y., and K. H. J., Front Pharmacol. (2017); 8: 817.
Pahan, K., Improvement of Brain Function by a Lipid-Lowering Factor, (2017) https://www.rush.edu/sites/default/files/2017RushNeuroscienceReview.pdf#page=20
Pahan, K. (2006) Lipid-lowering drugs. Cell Mol Life Sci, 63, 1165-1178.

Pahan, K., Jana, M., Liu, X., Taylor, B. S., Wood, C. and Fischer, S. M. (2002) Gemfibrozil, a lipidlowering drug, inhibits the induction of nitric-oxide synthase in human astrocytes. J Biol Chem, 277, 45984-45991.
Pavuluri, P., Vadakedath, S., Gundu, R., Uppulety, S., and Kandi, V., Cureus (2017) January; 9(1): e949.
Prorok, T., Malabendu, J., Patel, D., and Pahan, K., Neurochem Research (2019) https://doi.org/10.1007/s11064-018-02705-0.
Puranam, K., Qian, W. H., Nikbakht, K., Venable, M., Obeid, L., Hannun, Y. and Boustany, R. M. (1997) Upregulation of Bcl-2 and elevation of ceramide in Batten disease. Neuropediatrics, 28, 37-41.
Robins, S. J., Collins, D., Wittes, J. T. et al. (2001) Relation of gemfibrozil treatment and lipid levels with major coronary events: VA-HIT: a randomized controlled trial. JAMA, 285, 1585-1591.
Roleira, F. M.; Siquet, C.; Orru, E.; Garrido, E. M.; Garrido, J.; Milhazes, N.; Podda, G.; Paiva-Martins, F.; Reis, S.; Carvalho, R. A.; et al. Lipophilic phenolic antioxidants: Correlation between antioxidant profile, partition coefficients and redox properties. Bioorg. Med. Chem. 2010, 18, 5816-5825.
Roy, A. and Pahan, K. (2009) Gemfibrozil, stretching arms beyond lipid lowering. Immunopharmacol Immunotoxicol, 31, 339-351.
Rubins, H. B. and Robins, S. J. (1992) Effect of reduction of plasma triglycerides with gemfibrozil on high-density-lipoprotein-cholesterol concentrations. J Intern Med, 231, 421-426.
Rubins, H. B., Robins, S. J., Collins, D. et al. (1999) Gemfibrozil for the secondary prevention of coronary heart disease in men with low levels of high-density lipoprotein cholesterol. Veterans Affairs High-Density Lipoprotein Cholesterol Intervention Trial Study Group. N Engl J Med, 341, 410-418.
Shaw, Alan R. & Feinberg, Mark B., Clinical immunology (4th ed., 2013).
Sleat, D. E., Donnelly, R. J., Lackland, H., Liu, C. G., Sohar, I., Pullarkat, R. K., and Lobel, P. (1997) Science 277, 1802-1805.
Vines, D. J., and Warburton, M. J. (1999) FEBS Lett 443, 131-135.
Viglio, S., Marchi, E., Wisniewski, K., Casado, B., Cetta, G., and Iadarola, P. (2001) Electrophoresis 22, 2343-2350.
Walus, M., Kida, E., and Golabek, A. A. (2010) Hum Mutat 31, 710-721.

1. A method for treatment of a lysosomal storage disease comprising administering to a subject in need thereof a first composition comprising a therapeutically effective amount of a gene encoding for a lysosomal enzyme and a second composition comprising a therapeutically effective amount of a pharmaceutical agent.

2. The method of claim 1, wherein the first composition is administered intra-nasally.

3. The method of claim 1, wherein the gene is delivered across the blood, brain barrier.

4. The method of claim 1, wherein the first composition is administered about once every 7-30 days.

5. The method of claim 1, wherein the first composition comprises a viral vector comprising the gene encoding for a lysosomal enzyme.

6. The method of claim 5, wherein the viral vector is an adenovirus-associated viral vector.

7. The method of claim 1, wherein the gene comprises ppt1, cln2, cln3, galc, or hexa.

8. The method of claim 1, wherein the lysosomal enzyme comprises palmitoyl-protein thioesterase-1, tripeptidyl peptidase 1, galactosylceramide, battenin or hexosaminidase A.

9. (canceled)

10. (canceled)

11. (canceled)

12. (canceled)

13. (canceled)

14. The method of claim 1, wherein the pharmaceutical agent comprises cinnamic acid, oleamide or fibrate.

15. The method of claim 14, wherein the fibrate is gemfibrozil or fenofibrate.

16. The method of claim 1, wherein the second composition further comprises a therapeutically effective amount of all-trans retinoic acid.

17. The method of claim 1, wherein the therapeutically effective amount of the pharmaceutical agent is lower when the pharmaceutical agent is administered in combination with all-trans retinoic acid than when the pharmaceutical agent is delivered without all-trans retinoic acid.

18. The method of claim 1, wherein the second composition is administered orally.

19. The method of claim 1, wherein second composition is administered once daily.

20. The method of claim 1, wherein administering the first composition and the second composition provides a greater therapeutic effect in the subject than administration of the first composition or the second composition alone.

21. The method of claim 1, wherein the lysosomal storage disorder is selected from the group consisting of late-infantile Batten disease, juvenile Batten disease, Krabbe disease, Tay-Sachs disease, Niemann-Pick disease, Fabry disease, Farber disease and Gaucher disease.

22. The method of claim 1, wherein the first composition is administered intra-nasally and the second composition is administered orally.

23. (canceled)

24. (canceled)

25. (canceled)

26. (canceled)

27. The method of claim 1, wherein administration of the first composition increases lifespan of the subject in need thereof by about 100 days.

28. The method of claim 1, wherein the lifespan of the subject in need thereof increases by at least 100 days.
